(19) United States
(12) Patent Application Publication
(10) Pub. No.: US 2026/0233023 A1
(43) Pub. Date: Aug. 13, 2026

(54) WEARABLE THERAPEUTIC DEVICE

(71) Applicant: Therabody, Inc., Los Angeles, CA (US)

(72) Inventors: Mandar DESHMUKH, Los Angeles, CA (US); Benjamin NAZARIAN, Los Angeles, CA (US); Jason WERSLAND, Los Angeles, CA (US); Timothy ROBERTS, Los Angeles, CA (US); Jason MORRIS, Los Angeles, CA (US)

(21) Appl. No.: 19/051,904

(22) Filed: Feb. 12, 2025

(51) Int. Cl. A61N 5/06; A61F 7/00; A61H 23/02

(52) U.S. Cl. CPC A61N 5/0613; A61F 7/007; A61H 23/0254; A61F 2007/0027; A61F 2007/0086; A61H 2201/0207; A61H 2201/0221; A61H 2201/10; A61H 2201/1623; A61H 2201/165; A61H 2205/081; A61N 2005/0645; A61N 2005/0652; A61N 2005/0659; A61N 2005/066

(57) ABSTRACT

A wearable therapeutic device comprises a strap configured to be worn around a lumbar region of a back of a user. The strap comprises a central portion, a first end portion extending from a first side of the central portion, and a second end portion extending from a second side of the central portion opposite the first side of the central portion. A first longitudinal axis of the first end portion and a second longitudinal axis of the second end portion are coaxial with each other, and a central longitudinal axis of the central portion is located further from a head of the user than the first longitudinal axis and the second longitudinal axis when the strap is worn by the user. At least one vibration element, a heating element, a near-infrared light source, and a far-infrared light source are each located in the central portion.

### TECHNICAL FIELD

The present disclosure relates to a wearable therapeutic device, more specifically, a wearable therapeutic strap to provide various therapy modalities to a lower back of a user.

### BACKGROUND

Therapeutic straps can be used for providing various treatment modalities to a user for various purposes (for example, recovery from exercise, recovery from injury, regular maintenance, etc.). Treatment modalities can include heat therapy, vibration therapy, and near infrared (NIR) light therapy, among others. However, typical therapeutic straps do not provide far infrared (FIR) light therapy, which can travel deeper into the tissue of the user than NIR light therapy to provide additional recovery benefits (for example, pain relief, improved blood circulation, inflammation reduction, and tissue repair). Thus, there is a need for a therapeutic strap that combines heat therapy, vibration therapy, NIR light therapy, and FIR light therapy.

Additionally, typical therapeutic straps can provide treatment for only one area of the back of the user (for example, a thoracic area, a lumbar area, etc.). However, a user may often need to treat multiple areas simultaneously. For example, users may need to treat both the lumbar and sacral regions (for example, the lumbosacral region) of the back simultaneously. Typical therapeutic straps do not provide the ability to treat the lumbar and sacral regions of the back at the same time because the shape of the lumbosacral region can limit the effectiveness of treatment in at least one of the lumbar or sacral regions. Thus, there is a need for a therapeutic strap that can effectively treat both the lumbar and sacral regions of the back of a user at the same time.

The background description disclosed anywhere in this patent application includes information that may be useful in understanding the present disclosure. It is not an admission that any of the information provided herein is prior art or relevant to the presently claimed concepts, or that any publication specifically or implicitly referenced is prior art.

### SUMMARY

Described herein is a wearable therapeutic device that can be worn by a user and provide heat therapy, vibration therapy, NIR light therapy, and FIR light therapy. In some embodiments, the therapeutic device can provide these therapies simultaneously. In some embodiments, the therapeutic device can provide these therapies separately. In some embodiments, the therapeutic device can provide these therapies in various combinations of two, three, or all four therapies. The therapeutic device can be worn around the back of the user to provide these therapies to the lumbar and sacral regions of the back at the same time.

In one embodiment, a wearable therapeutic device comprises a strap configured to be worn around a lumbar region of a back of a user. The strap can comprise a central portion comprising a central longitudinal axis extending approximately perpendicular to a spine of the user when the strap is being worn by the user. The strap can comprise a first end portion extending from a first side of the central portion and comprising a first longitudinal axis extending approximately perpendicular to the spine of the user when the strap is worn by the user. The strap can comprise a second end portion extending from a second side of the central portion and comprising a second longitudinal axis extending approximately perpendicular to the spine of the user when the strap is worn by the user. The first longitudinal axis and the second longitudinal axis are coaxial with each other, and the central longitudinal axis is located further from a head of the user than the first longitudinal axis and the second longitudinal axis when the strap is worn by the user. The wearable therapeutic device comprises at least one vibration element located in the central portion, a heating element located in the central portion, a near-infrared (NIR) light source located in the central portion, and a far-infrared (FIR) light source located in the central portion.

In another embodiment, a wearable therapeutic device comprises a strap to be worn around a lumbar region of a back of a user. The strap comprises a central portion, a first end portion extending from a first side of the central portion, and a second end portion extending from a second side of the central portion opposite the first side of the central portion. The wearable therapeutic device comprises at least one vibration element located in the central portion. The wearable therapeutic device comprises a NIR light source located in the central portion. The NIR light source is located closer to a spine of the user than the vibration element when the strap is worn by the user. The wearable therapeutic device comprises a carbon fiber heating layer located in the central portion and is located between the at least one vibration element and the NIR light source when the strap is worn by the user. The carbon fiber heating layer emits FIR light when the carbon fiber heating layer receives power from a power source in the strap.

In yet another embodiment, a wearable therapeutic device comprises a strap to be worn around a lumbar region of a back of a user. The strap comprises a central portion comprising a top edge and a bottom edge, the top edge being located closer to a head of the user when the strap is worn by the user. The strap comprises a first end portion extending from a first side of the central portion and comprising a top edge and a bottom edge, the top edge being located closer to the head of the user when the strap is worn by the user. The strap comprises a second end portion extending from a second side of the central portion and comprising a top edge and a bottom edge, the top edge being located closer to the head of the user when the strap is worn by the user. The second end portion comprises a controller configured to be located in front of the user when the strap is worn by the user. A first distance between the top edge of the central portion and the top edges of the first end portion and the second end portion in a direction approximately parallel to a spinal axis of the user is smaller than a second distance between the bottom edge of the central portion and the bottom edges of the first end portion and the second end portion in the direction approximately parallel to the spinal axis of the user. The wearable therapeutic device comprises at least one vibration element located in the central portion and is electrically coupled to the controller. The wearable therapeutic device comprises a heating element located in the central portion and is electrically coupled to the controller. The wearable therapeutic device comprises a NIR light source located in the central portion and is electrically coupled to the controller. The wearable therapeutic device comprises a FIR light source located in the central portion and is electrically coupled to the controller. The controller is configured to operate the at least one vibration element, the heating element, the NIR light source, and the FIR light source.

Further features and advantages, as well as the structure and operation of various aspects, are described in detail below with reference to the accompanying drawings. It is noted that the specific aspects described herein are not intended to be limiting. Such aspects are presented herein for illustrative purposes only. Additional aspects will be apparent to persons skilled in the relevant art(s) based on the teachings contained herein.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the pertinent art to make and use the disclosure.

FIGS. 1A-1B are front and rear views, respectively, of a user wearing a therapeutic device according to embodiments of the present disclosure.

FIG. 2 is a front view of a therapeutic device according to embodiments of the present disclosure.

FIG. 3 is a rear view of the therapeutic device of FIG. 2 according to embodiments of the present disclosure.

FIG. 4A is a rear view of the therapeutic device of FIG. 2 with an outer layer removed according to embodiments of the present disclosure.

FIGS. 4B-4C show carbon fiber designs of the therapeutic device of FIG. 4A according to embodiments of the present disclosure.

FIG. 5 is a rear view of the areas of a user targeted by the therapies of the therapeutic device of FIG. 2 according to embodiments of the present disclosure.

FIG. 6 is a cross-sectional view of the therapeutic device of FIG. 4A across A-A according to embodiments of the present disclosure.

FIG. 7 is a cross-sectional view of the therapeutic device of FIG. 4A across B-B according to embodiments of the present disclosure.

FIG. 8 is a cross-sectional view of the therapeutic device of FIG. 4A across C-C according to embodiments of the present disclosure.

FIG. 9 is a perspective view of a lumbar support element according to embodiments of the present disclosure.

FIG. 10 is an illustration of a controller according to embodiments of the present disclosure.

FIG. 11 is a block diagram of the controller of FIG. 10 according to embodiments of the present disclosure.

FIG. 12 is a flowchart of a method to determine a treatment temperature according to embodiments of the present disclosure.

In the drawings, like reference numbers generally indicate identical or similar elements. Additionally, generally, the left-most digit(s) of a reference number identifies the drawing in which the reference number first appears.

### DETAILED DESCRIPTION

The following description and drawings are illustrative and are not to be construed as limiting. Numerous specific details are described to provide a thorough understanding of the disclosure. However, in certain instances, well-known or conventional details are not described in order to avoid obscuring the description. References to one or more aspects in the present disclosure can be, but not necessarily are references to the same aspect; and, such references mean at least one of the aspects. If a component is not shown in a drawing then this provides support for a negative limitation in the claims stating that that component is “not” present. However, the above statement is not limiting and in another aspect, the missing component can be included in a claimed aspect.

Reference in this specification to “one embodiment,” “an embodiment,” “a preferred aspect” or any other phrase mentioning the word “embodiment” means that a particular feature, structure, or characteristic described in connection with the embodiment is included in at least one embodiment of the-disclosure and also means that any particular feature, structure, or characteristic described in connection with one aspect can be included in any embodiment or can be omitted or excluded from any aspect. The appearances of the phrase “in one embodiment” in various places in the specification are not necessarily all referring to the same aspect, nor are separate or alternative aspects mutually exclusive of other aspects. Moreover, various features are described which may be exhibited by some embodiments and not by others and may be omitted from any aspect. Furthermore, any particular feature, structure, or characteristic described herein may be optional. Similarly, various requirements are described which may be requirements for some embodiments but not other embodiments. Where appropriate any of the features discussed herein in relation to one embodiment of the disclosure may be applied to another embodiment of the disclosure. Similarly, where appropriate any of the features discussed herein in relation to one embodiment of the disclosure may be optional with respect to and/or omitted from that embodiment of the disclosure or any other embodiment of the disclosure discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various aspects given in this specification.

Without intent to further limit the scope of the disclosure, examples of instruments, apparatus, methods and their related results according to the aspects of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present disclosure.

The terms “connected” or “coupled” and related terms are used in an operational sense and are not necessarily limited to a direct connection or coupling. The term “thermally coupled” means coupled in a way capable of conducting heat, and the term “thermally insulated” means separated by a substance that deters heat transfer.

The term “flexible” generally means bendable and adaptable under relatively little force. In the context of various aspects of the present disclosure, flexible is intended to describe the dynamic conforming nature of the personal temperature controlled device to the general shape of a portion of a person's body, such as wrist, ankle, neck, shoulder, back, chest, forehead, rib cage, arch, temple, palm, etc., directly or indirectly in contact with or otherwise engaging a surface of the personal temperature-controlled device. In addition, the term “approximately” is generally used to modify a numerical value above and below the set value by a variation of +/−10%.

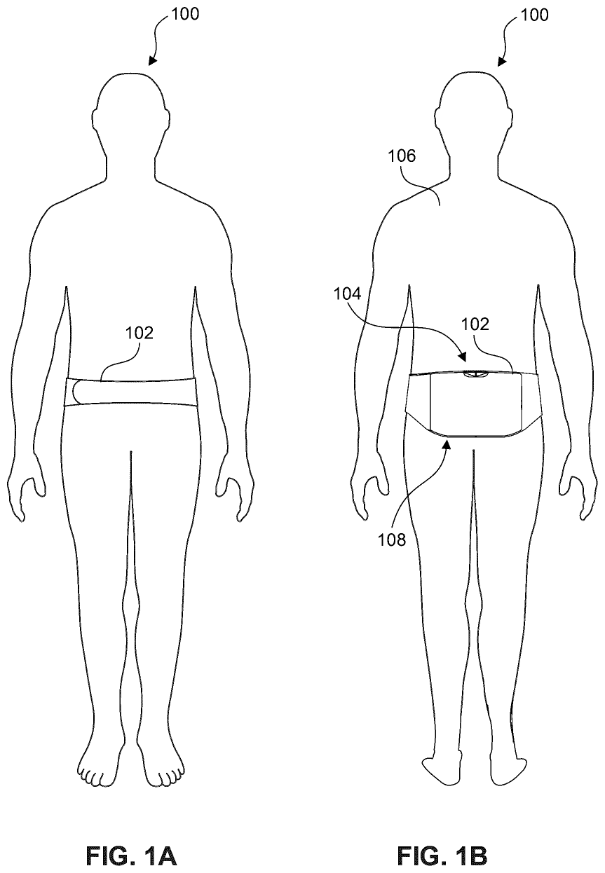

FIGS. 1A-1B are front and rear views, respectively, of a user 100 wearing a therapeutic device 102 according to embodiments of the present disclosure. The therapeutic device 102 can be secured to the user 100 such that the user 100 does not have to hold the device on the body of the user 100 (for example, opposing end portions of the therapeutic device 102 can be wrapped around the user 100 and secured to each other to secure the therapeutic device 102 to the user 100). As shown, the therapeutic device 102 can be worn around a lumbar region 104 of a back 106 of the user 100. The therapeutic device 102 can be worn around a sacral region 108 of the back 106. In some embodiments, the therapeutic device 102 is shaped such that the therapeutic device 102 is configured to be worn around the lumbar region 104 and the sacral region 108 (for example, a lumbosacral region) simultaneously.

The therapeutic device 102 is configured to provide various therapy modalities to the lumbar region 104 and/or the sacral region 108. In some embodiments, the therapeutic device 102 can provide vibration therapy to the lumbar region 104 and/or the sacral region 108. In some embodiments, the therapeutic device 102 can provide heat therapy to the lumbar region 104 and/or the sacral region 108. In some embodiments, the therapeutic device 102 can provide light therapy (for example, one or more of NIR light therapy or FIR light therapy) to the lumbar region 104 and/or the sacral region 108. In some embodiments, the therapeutic device 102 can provide two or more of the aforementioned therapy modalities to the lumbar region 104 and/or the sacral region 108 simultaneously.

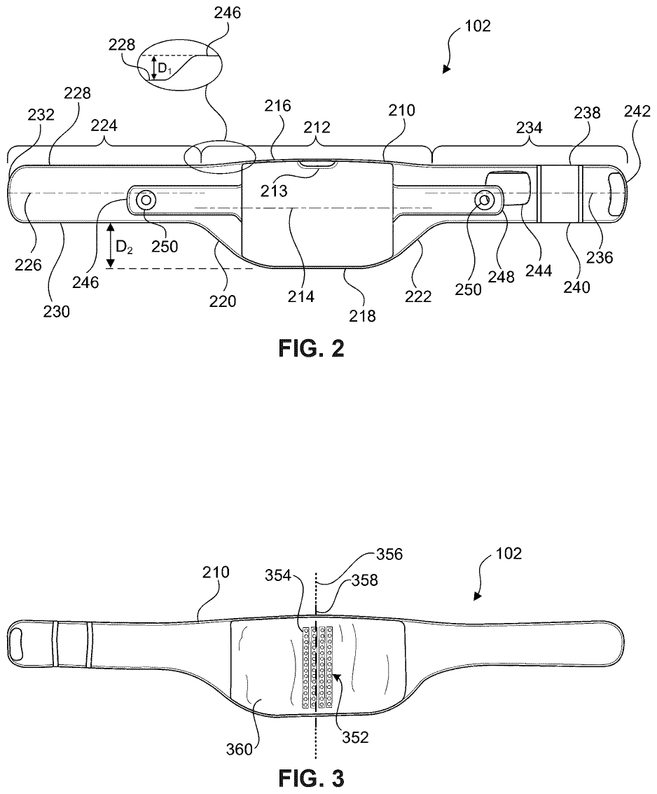

FIG. 2 is a front view of the therapeutic device 102 according to embodiments of the present disclosure. The therapeutic device 102 can comprise a strap 210 configured to be worn around the lumbar region 104. In some embodiments, the strap 210 can comprise a central portion 212. The central portion 212 can comprise a central longitudinal axis 214 that extends approximately perpendicular to a spine of the user 100 (shown in FIG. 1) when the strap 210 is worn by the user 100. In some embodiments, the central portion 212 can be sized to cover at least a portion of the lumbar region 104 and a least a portion of the sacral region 108 of the back 106 of the user 100. In some embodiments, the central portion 212 can comprise a top edge 216 located opposite a bottom edge 218, where the top edge 216 is located closer to a head of the user 100 when the strap 210 is worn by the user 100. In some embodiments, the central portion 212 can have a height (for example, a distance between the top edge 216 and the bottom edge 218) of greater than or equal to 160 millimeters (mm) and less than or equal to 250 mm. In some embodiments, the central portion 212 can comprise a first side 220 located opposite a second side 222. In some embodiments, the central portion 212 can have a width (for example, a distance between the first side 220 and the second side 222) of greater than or equal to 300 mm and less than or equal to 550 mm. In some embodiments, the central portion 212 may comprise an opening 213 in which a lumbar support element (shown in FIG. 9) may be inserted to provide additional lumbar support to the user 100 and help with the user's posture.

In some embodiments, the strap 210 can comprise a first end portion 224 extending from the first side 220 of the central portion 212. The first end portion 224 can comprise a first longitudinal axis 226 that extends approximately perpendicular to the spine of the user 100 when the strap 210 is worn by the user 100. The first end portion 224 can comprise a top edge 228 located opposite a bottom edge 230, where the top edge 228 is located closer to the head of the user 100 when the strap 210 is worn by the user 100. In some embodiments, the first end portion 224 can have a height (for example, a distance between the top edge 228 and the bottom edge 230) of greater than or equal to 80 mm and less than or equal to 110 mm. In some embodiments, the first end portion 224 can comprise a first end 232 located opposite the first side 220. In some embodiments, the first end portion 224 can have a width (for example, a distance between the first side 220 and the first end 232) of greater than or equal to 350 mm and less than or equal to 450 mm.

In some embodiments, the strap 210 can comprise a second end portion 234 extending from the second side 222 of the central portion 212. The second end portion 234 can comprise a second longitudinal axis 236 that extends approximately perpendicular to the spine of the user 100 when the strap 210 is worn by the user 100. The second end portion 234 can comprise a top edge 238 located opposite a bottom edge 240, where the top edge 238 is located closer to the head of the user 100 when the strap 210 is worn by the user 100. In some embodiments, the second end portion 234 can have a height (for example, a distance between the top edge 238 and the bottom edge 240) of greater than or equal to 80 mm and less than or equal to 110 mm. In some embodiments, the height of the second end portion 234 can be approximately equal to the height of the first end portion 224. In some embodiments, the height of the second end portion 234 and the height of the first end portion 224 can be different. In some embodiments, the second end portion 234 can comprise a second end 242 located opposite the second side 222. In some embodiments, the second end portion 234 can have a width (for example, a distance between the second side 222 and the second end 242) of greater than or equal to 350 mm and less than or equal to 450 mm). In some embodiments, the width of the second end portion 234 can be approximately equal to the width of the first end portion 224. In some embodiments, the width of the second end portion 234 and the width of the first end portion 224 can be different.

In some embodiments, the first longitudinal axis 226 and the second longitudinal axis 236 can be approximately coaxial with each other. In such embodiments, the top edge 228 can be approximately aligned with the top edge 238, and the bottom edge 230 can be approximately aligned with the bottom edge 240. In some embodiments, the central longitudinal axis 214 can be located further from the head of the user 100 than the first longitudinal axis 226 and the second longitudinal axis 236 when the strap 210 is worn by the user 100. In some embodiments, the top edge 216 is not aligned with the top edge 228 or the top edge 238. In some embodiments, the bottom edge 218 is not aligned with the bottom edge 230 or the bottom edge 240.

In some embodiments, a distance D₁ can be a vertical distance (for example, a distance in a direction approximately parallel to the spine of the user 100) between the top edge 216 and the top edge 228. In some embodiments, a vertical distance between the top edge 216 and the top edge 238 can be the same as the distance D₁. Accordingly, the following description of D₁ can be applied to the vertical distance between the top edge 216 and the top edge 238. In some embodiments, a distance D₂ can be a vertical distance between the bottom edge 230 and the bottom edge 218. In some embodiments, a vertical distance between the bottom edge 240 and the bottom edge 218 can be the same as the distance D₂. Accordingly, the following description of D₂ can be applied to the vertical distance between the bottom edge 240 and the bottom edge 218. In some embodiments, D₁ is smaller than D₂ such that the bottom edge 218 is further from the first longitudinal axis 226 and the second longitudinal axis 236 than the top edge 216. As described, the strap 210 is asymmetric about the central longitudinal axis 214. The strap 210 is also asymmetric about the first longitudinal axis 226 and the second longitudinal axis 236. The asymmetric arrangement of the strap 210 allows the central portion 212 to contact both the lumbar region 104 and the sacral region 108 of the back 106 of the user 100 when the strap 210 is worn by the user 100.

For example, the user 100 can secure the strap 210 to the body of the user 100 by placing the central portion 212 in contact with the lumbar region 104 and the sacral region 108, and wrapping the first end portion 224 and the second end portion 234 around a waist of the user 100. In some embodiments, the first end portion 224 and the second end portion 234 can be coupled to each other via, for example, a hook and loop connection. For example, one of the first end portion 224 or the second end portion 234 can comprise one of a hook portion or a loop portion, and the other of the first end portion 224 or the second end portion 234 can comprise the other of the hook portion or the loop portion. When the strap 210 is secured to the body of the user, the central portion 212 at least partially covers both the lumbar region 104 and the sacral region 108 of the back 106 of the user 100.

In some embodiments, the strap 210 can comprise a first tightening strap 246 extending from the central portion 212 toward the first end 232 and a second tightening strap 248 extending from the central portion 212 toward the second end 242. The first tightening strap 246 and the second tightening strap 248 can be used to tighten the securement between the strap 210 and the user 100. For example, each of the first tightening strap 246 and the second tightening strap 248 can comprise an elastic material that can stretch. The user 100 can stretch each of the first tightening strap 246 and the second tightening strap 248 and secure the first tightening strap 246 to the first end portion 224 (via, for example, a hook and loop connection) and secure the second tightening strap 248 to the second end portion 234 (via, for example, a hook and loop connection). In some embodiments, the first tightening strap 246 and the second tightening strap 248 may allow the user 100 to adjust the fit of the strap 210 around the user's waist and hip area for a tighter and more snug fit. By securing the first and second tightening straps 246, 248 in a tighter fit when worn by the user 100, the therapeutic elements in the strap 210 (e.g., vibration element(s), FIR light source, NIR light source, heating element(s), etc.) can make better contact with the lumbar region 104 and/or sacral region 108 of the user 100 in order to improve the delivery of therapy to the user 100 and enhance the user's therapeutic experience and perception of the strap's therapy modalities provided to the user 100. In some embodiments, the first tightening strap 246 and the second tightening strap 248 can each comprise an opening 250 for the user 100 to grasp (for example, by extending one or more fingers or a thumb through the opening 250). In some embodiments, the openings 250 may allow the user 100 to grasp the first and second tightening straps 246, 248 more easily and pull the straps in a tighter fit. In some embodiments, the openings 250 can be omitted. In some embodiments, the strap 210 may include an additional extension strap that may be coupled to the first end portion 224 and the second end portion 234 by a hook and loop connection. By coupling the additional extension strap to the strap 210 and securing the first and second tightening straps 246, 248, the strap 210 may be configured to accommodate different body shapes and sizes of various users.

In some embodiments, the second end portion 234 can comprise a controller 244. The controller 244 can be configured to operate each of the therapy modalities provided by the therapeutic device 102. In some embodiments, the controller 244 is configured to be located in front of the user 100 when the strap 210 is worn by the user 100. For example, when the user 100 wraps the second end 242 around the waist of the user 100 and secures the second end 242 to the first end 232, the controller 244 is located in front of the user 100 such that the user 100 can look down and view the controller 244 such that the user 100 can operate the therapeutic device 102. Accordingly, the first end 232 can be configured to be located between the user 100 and the second end 242 when the strap 210 is worn by the user 100 such that the first end 232 does not cover the controller 244 or otherwise obscure the view of the controller 244 from the user 100.

FIG. 3 is a rear view of the therapeutic device 102 of FIG. 2 according to embodiments of the present disclosure. As shown, a NIR light source 352 is located in the central portion 212. NIR light has been shown to relieve pain, inflammation, and swelling in muscles and joints. More specifically, NIR light has been shown to relieve pain, inflammation, and swelling in tendinous regions (for example, where muscles are attached to bones). The NIR light source 352 can comprise at least one light emitting diode (LED) strip 354. In some embodiments, each LED strip 354 can comprise a group of LEDs arranged linearly, and each LED is configured to emit light at a wavelength of greater than or equal to 820 nanometers (nm) and less than or equal to 860 nm. In some embodiments, the at least one LED strip 354 can be oriented approximately vertically (for example, approximately parallel to the spine of the user) when the strap 210 is worn by the user. The NIR light source 352 can comprise an axis 356 that is approximately coaxial with a transverse axis 358 of the central portion 212 (for example, an axis approximately parallel to the spine of the user 100 when the strap 210 is being worn by the user 100 and extending through a midpoint between the first side 220 and the second side 222). In some embodiments, the NIR light source 352 can be electrically coupled to the controller 244 such that the controller 244 can operate the NIR light source 352.

As shown in FIG. 3, the NIR light source 352 is exposed. For example, the NIR light source 352 can be located on an outer surface of a cover 360 such that the NIR light source 352 can contact the user directly. More specifically, the NIR light source 352 can contact the skin of the user 100 when the therapeutic device 102 is worn under the clothing of the user 100, or the NIR light source 352 can contact the clothing of the user 100 when the therapeutic device 102 is worn on top of the clothing of the user 100. In some embodiments, the cover 360 is configured to cover the other therapy modalities to secure the other therapy modalities within the strap 210.

In some embodiments, the NIR light source 352 can be configured to emit a dose of NIR light based on an amount of time the NIR light source 352 operates. For example, the NIR light source 352 can emit a dose of NIR light greater than or equal to 50 Joules per square centimeter and less than or equal to 150 Joules per square centimeter when the NIR light source 352 operates for approximately 10 minutes. As another example, the NIR light source 352 can emit a dose of NIR light greater than 150 Joules per square centimeter and less than or equal to 300 Joules per square centimeter when the NIR light source 352 operates for approximately 20 minutes.

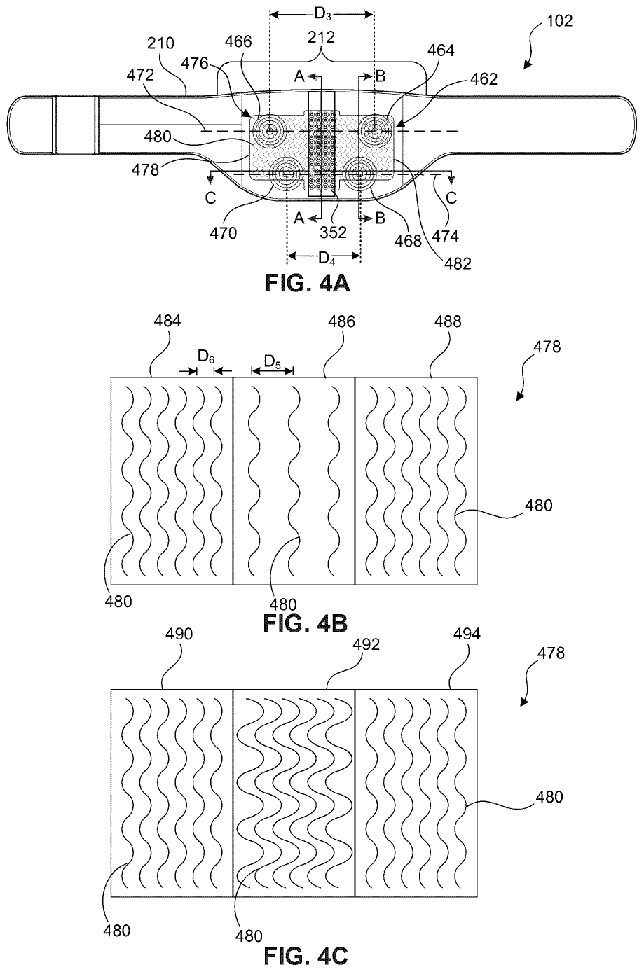

FIG. 4A is a rear view of the therapeutic device 102 of FIG. 2 with the cover 360 removed according to embodiments of the present disclosure. With the cover 360 removed, the remaining therapy modalities can be viewed, and are described below. As described with reference to FIG. 3, the therapeutic device 102 can comprise the NIR light source 352 located in the central portion 212. In some embodiments, the NIR light source 352 can be electrically coupled to the controller 244 such that the controller 244 can control operation of the NIR light source 352.

The therapeutic device 102 can comprise at least one vibration element located in the central portion 212. In some embodiments, the at least one vibration element can be electrically coupled to the controller 244 such that the controller 244 can control operation of the at least one vibration element. The at least one vibration element can be any type of device or system configured to generate vibrations such that vibration therapy can be applied to the back 106 of the user 100. For example, the at least one vibration element can generate vibrations by eccentrically rotating a weight about a central axis. Other known systems and devices can be used to generate the vibration therapy. In some embodiments, the at least one vibration element is configured to generate vibrations having a frequency of up to 100 Hertz (Hz). In some embodiments, the at least one vibration element is configured to generate vibrations having a frequency of greater than or equal to 50 Hz and less than or equal to 100 Hz.

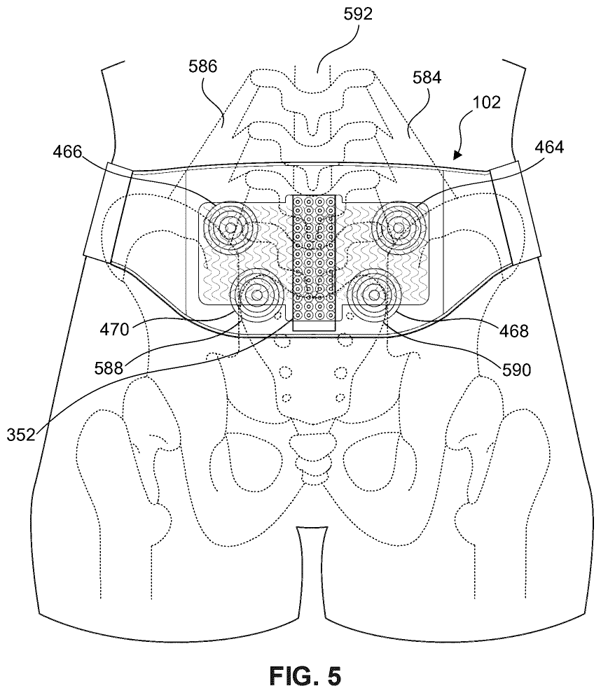

In some embodiments, the strap 210 can comprise vibration elements 462 arranged to provide vibration therapy to specific areas of the back 106 of the user 100. The vibration elements 462 can comprise a first vibration element 464, a second vibration element 466, a third vibration element 468, and a fourth vibration element 470. The first vibration element 464 and the second vibration element 466 can lie along an axis 472 that is approximately parallel to the central longitudinal axis 214. The first vibration element 464 and the second vibration element 466 can be approximately equidistant from the transverse axis 358. The third vibration element 468 and the fourth vibration element 470 can lie along an axis 474 that is approximately parallel to the central longitudinal axis 214. The axis 472 can be located closer to the head of the user 100 than axis 474 when the strap 210 is worn by the user 100. Thus, the first vibration element 464 and the second vibration element 466 are located closer to the head of the user 100 than the third vibration element 468 and the fourth vibration element 470 when the strap 210 is worn by the user 100. A distance D₃ between the first vibration element 464 and the second vibration element 466 (for example, a distance along the axis 472) can be greater than a distance D₄ between the third vibration element 468 and the fourth vibration element 470 (for example, a distance along the axis 474). Arranged as described, the vibration elements 462 can provide vibration therapy to areas of the back 106 of the user 100. For example, the vibration elements 462 can provide vibration therapy to one or more of the quadratus lumborum muscles and/or one or more of the sacroiliac joints. Providing vibration therapy to specific areas of the back 106 of the user 100 is further described with reference to FIG. 5.

In some embodiments, the strap 210 can comprise a heating element 476 located in the central portion 212. Heat therapy has been shown to relieve musculoskeletal pain generally, and lower back pain specifically. The heating element 476 can be configured to provide heat therapy to the back 106 of the user 100. In some embodiments, the heating element 476 can be electrically coupled to the controller 244 such that the controller 244 can control operation of the heating element 476. In some embodiments, the heating element 476 can comprise a carbon fiber heating portion 478. The carbon fiber heating portion 478 can comprise carbon fiber strands 480 arranged in a pattern. In some embodiments, the carbon fiber heating portion 478 may comprise a single layer or a double layer of carbon fiber strands 480. In some embodiments, the carbon fiber strands 480 can be arranged in a sinusoidal pattern. In some embodiments, the carbon fiber strands 480 can be arranged in a vertical sinusoidal pattern (as shown in FIGS. 4A-4C), in a horizontal sinusoidal pattern, or in a combination of both vertical and horizontal sinusoidal patterns. In some embodiments, arranging the carbon fiber strands 480 in a horizontal sinusoidal pattern may maximize the heating surface area of the carbon fiber heating portion 478 and reduce non-heating elements (such as wiring) in the surface area of the carbon fiber heating portion 478.

In some embodiments, the sinusoidal patterns of the carbon fiber strands 480 may be arranged with different distances between the wave patterns and different sinusoidal amplitudes in different sections of the carbon fiber heating portion 478, as shown in FIGS. 4B and 4C. FIGS. 4B and 4C show carbon fiber designs of the therapeutic device 102 of FIG. 4A according to embodiments of the present disclosure. As shown in FIG. 4B, the carbon fiber heating portion 478 may comprise a first section 484, a second section 486, and a third section 488, with carbon fiber strands 480 arranged in each section. The pattern of the carbon fiber strands 480 in first section 484 may be the same as the pattern of the carbon fiber strands 480 in third section 488, whereas the pattern of the carbon fiber strands 480 in the second section 486 may differ with respect to the distance between each carbon fiber strand 480. In particular, the distance D₅ between each carbon fiber strand 480 in the second section 486 may be greater than the distances D₆ between each carbon fiber strand 480 in sections 484 and 488. FIG. 4C shows carbon fiber heating portion 478 comprising a first section 490, a second section 492, and a third section 494, with carbon fiber strands 480 arranged in each section. As shown in FIG. 4C, the pattern of the carbon fiber strands 480 in first section 490 may be the same as the pattern of the carbon fiber strands 480 in third section 494, whereas the pattern of the carbon fiber strands 480 in the second section 492 may differ with respect to sinusoidal amplitudes or wave amplitudes. In particular, the carbon fiber strands 480 in the second section 492 may have a higher wave amplitude than the wave amplitudes of the carbon fiber strands 480 in sections 490 and 494.

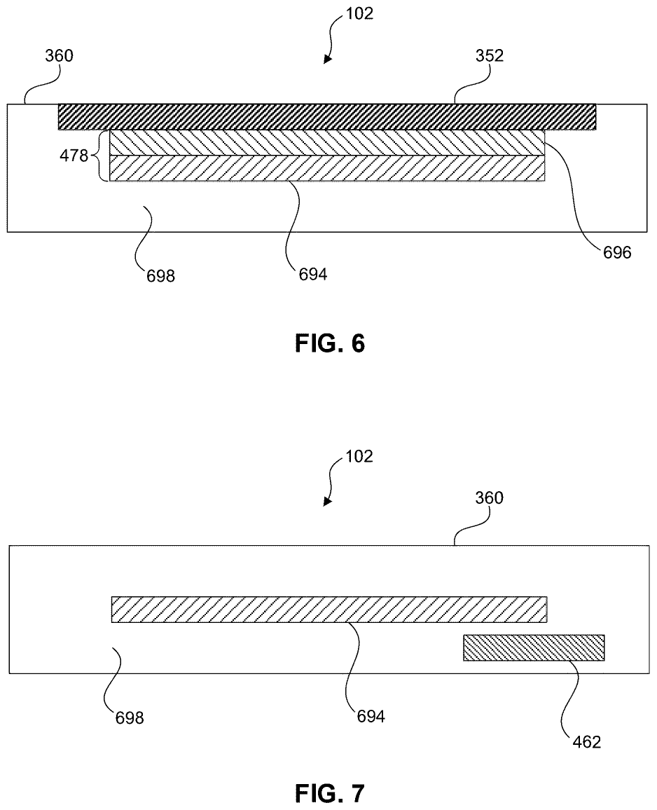

In some embodiments, the carbon fiber strands 480 can be arranged in a switchback pattern. When a current flows through the carbon fiber strands 480 (for example, when a power source provides power to the carbon fiber heating portion 478 to cause a current to flow through the carbon fiber strands 480), the temperature of the carbon fiber strands 480 increases to provide heat to the back 106 of the user 100. In some embodiments, the carbon fiber heating portion 478 is configured to evenly heat both the lumbar region 104 and the sacral region 108 (for example, the lumbosacral region) of the back 106 of the user 100. In some embodiments, the carbon fiber heating portion 478 can comprise multiple layers to achieve the even heating of the lumbosacral region. Such embodiments are described with reference to FIGS. 6-8.

In some embodiments, the heating element 476 can additionally or alternatively comprise graphene. Graphene may be integrated as a thin, flexible film, which can be incorporated easily into devices that prioritize sleek designs, user comfort, and lightweight construction. For example, a thin graphene-based heater layer can be embedded under a device without significantly altering the device's form.

In small devices, where uniform and quick heat distribution is critical, graphene films can heat up more consistently and at lower power consumption compared to larger carbon fiber elements. This could provide a more controlled and responsive heating mechanism.

However, because graphene is extremely thin and delicate, it often necessitates structural support by a substrate material (like flexible polymers, glass, or ceramics) and may need protective coatings to ensure durability and prevent oxidation or contamination. With carbon fiber, structural stability is often inherent to the fibers themselves.

In some embodiments, the strap 210 can comprise a FIR light source 482 located in the central portion 212. FIR light has been shown to have a therapeutic effect on the body. Specifically, FIR light has been shown to penetrate deeper into tissues than other therapies to provide more complete relief of pain and inflammation. The FIR light source 482 can be electrically coupled to the controller 244 such that the controller 244 can control operation of the FIR light source 482. In some embodiments, the FIR light source 482 and the carbon fiber heating portion 478 can be the same component. For example, the carbon fiber strands 480 are configured to emit FIR light when a current flows through the carbon fiber strands 480 (for example, when a power source provides power to the carbon fiber heating portion 478 to cause a current to flow through the carbon fiber strands 480). Thus, the carbon fiber heating portion 478 and the FIR light source 482 can be used interchangeably herein.

In some embodiments, the amount of FIR light emitted by the carbon fiber heating portion 478 may depend on the size and/or surface area of the carbon fiber heating portion 478. By controlling the levels of current and voltage provided to the carbon fiber heating portion 478 over predetermined periods of time, the therapeutic device 102 may provide different dosages of FIR radiation to the back 106 of the user 100. In some embodiments, the carbon fiber heating portion 478 may be configured to emit FIR light at a wavelength of greater than or equal to 4 micrometers (μm) and less than or equal to 16 micrometers (μm). In some embodiments, increasing the surface area of the carbon fiber heating portion 478 with dose-specific inputs (e.g., specific current and voltage levels) may decrease the wavelength of the FIR light emitted from the carbon fiber heating portion 478, whereas decreasing the surface area of the carbon fiber heating portion 478 with dose-specific inputs may increase the wavelength of the FIR light emitted from the carbon fiber heating portion 478.

In some embodiments, an amount of FIR light emitted by the FIR light source 482 depends on the temperature of the carbon fiber heating portion 478. For example, when the carbon fiber heating portion 478 reaches a first temperature (for example, approximately 39 degrees Celsius) and is maintained at the first temperature for a first duration (for example, greater than or equal to 10 minutes and less than or equal to 20 minutes), the FIR light source 482 is configured to emit a first dose of FIR light (for example, greater than or equal to 8 Joules per square centimeter and less than or equal to 10 Joules per square centimeter). As another example, when the carbon fiber heating portion 478 reaches a second temperature (for example, approximately 42 degrees Celsius) and is maintained at the second temperature for a second duration (for example, greater than or equal to 10 minutes and less than or equal to 20 minutes), the FIR light source 482 is configured to emit a second dose of FIR light (for example, greater than or equal to 10 Joules per square centimeter and less than or equal to 12 Joules per square centimeter).

FIG. 5 is a rear view of the areas of a user targeted by the therapies of the therapeutic device 102 of FIG. 2 according to embodiments of the present disclosure. The therapeutic device 102 is shown as semi-transparent in FIG. 5 for purposes of explanation of the targeted therapies provided by the therapeutic device 102. For example, the first vibration element 464 is configured to be aligned with the right quadratus lumborum muscle 584 and the second vibration element 466 is configured to be aligned with the left quadratus lumborum muscle 586 when the strap 210 is worn by the user 100. The quadratus lumborum muscles have been found to be a significant contributor to back pain and stiffness. Thus, the first vibration element 464 and the second vibration element 466 are configured to provide vibration therapy to the quadratus lumborum muscles to relieve pain and stiffness. The third vibration element 468 is configured to be aligned with the left sacroiliac joint 588 and the fourth vibration element 470 is configured to be aligned with the right sacroiliac joint 590 when the strap 210 is worn by the user 100. The sacroiliac joints are also a common source of lower back pain. Thus, the third vibration element 468 and the fourth vibration element 470 are configured to provide vibration therapy to the sacroiliac joints. The NIR light source 352 is aligned with the spine 592 and is configured to provide NIR light therapy to the lumbosacral region of the spine 592 to relieve pain, inflammation, and swelling in the lumbosacral region of the spine 592. The carbon fiber heating portion 478 (and thus, the FIR light source 482), is shown to extend across the central portion 212 such that both heat therapy and FIR light therapy can be provided to the lumbosacral region of the back 106 of the user 100 (for example, the quadratus lumborum muscles, the sacroiliac joints, the spine, and other muscles, tendons, ligaments, and joints in the lumbosacral region).

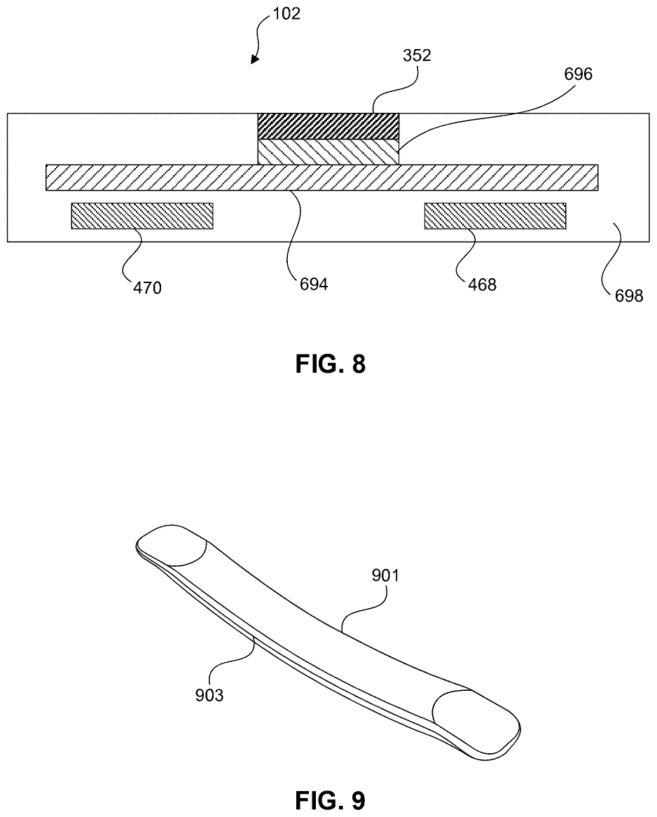

FIGS. 6-8 are cross-sectional views of the therapeutic device 102. FIG. 6 is a cross-sectional view of the therapeutic device 102 of FIG. 4A across A-A according to embodiments of the present disclosure. FIG. 7 is a cross-sectional view of the therapeutic device 102 of FIG. 4A across B-B according to embodiments of the present disclosure. FIG. 8 is a cross-sectional view of the therapeutic device 102 of FIG. 4A across C-C according to embodiments of the present disclosure. As shown, an outer surface of the NIR light source 352 can be aligned with the cover 360. In some embodiments, the outer surface of the NIR light source 352 can extend beyond the cover 360. The NIR light source 352 is located closer to the spine 592 than the vibration elements 462 (shown in FIGS. 7-8) and the carbon fiber heating portion 478 when the strap 210 is worn by the user 100. The carbon fiber heating portion 478 is located between the vibration elements 462 and the NIR light source 352 when the strap 210 is worn by the user 100. In some embodiments, the NIR light source 352, the carbon fiber heating portion 478, and the vibration elements 462 are secured by and/or at least partially surrounded by a housing 698 (for example, the cover 360 can be an outer portion of the housing 698). In some embodiments, the housing 698 can limit movement of the vibration elements 462 relative to the central portion 212 to direct the vibration therapy to the user 100.

The carbon fiber heating portion 478 can comprise a first carbon fiber heating layer 694 and a second carbon fiber heating layer 696. Each of the first carbon fiber heating layer 694 and the second carbon fiber heating layer 696 can comprise the carbon fiber strands 480 as previously described. The second carbon fiber heating layer 696 is located closer to the back 106 (and thus, the spine 592) of the user 100 when the strap 210 is worn by the user 100. Accordingly, the second carbon fiber heating layer 696 is located between the first carbon fiber heating layer 694 and the NIR light source 352. In some embodiments, at least two of the NIR light source 352, the first carbon fiber heating layer 694, and the second carbon fiber heating layer 696 can be in physical contact with each other. In some embodiments, the NIR light source 352, the first carbon fiber heating layer 694, and the second carbon fiber heating layer 696 can be physically separated from each other (for example, by portions of the housing 698). In either instance, the NIR light source 352 and the first carbon fiber heating layer 694 are positioned adjacent to each other, and the first carbon fiber heating layer 694 and the second carbon fiber heating layer 696 are positioned adjacent to each other. In some embodiments, the vibration elements 462 are separated from the group of the NIR light source 352, the first carbon fiber heating layer 694, and the second carbon fiber heating layer 696 to limit direct contact between those components and the vibration elements 462.

In some embodiments, the first carbon fiber heating layer 694 and the second carbon fiber heating layer 696 are different sizes. More specifically, in some embodiments, the first carbon fiber heating layer 694 can be larger than the second carbon fiber heating layer 696. For example, the first carbon fiber heating layer 694 can have a first surface area and the second carbon fiber heating layer 696 can have a second surface area, and the first surface area can be larger than the second surface area. More specifically, in some embodiments, the first surface area can be at least twice as large as the second surface area. In some embodiments, the second carbon fiber heating layer 696 can have approximately the same size and shape as the NIR light source 352. In some embodiments, the second carbon fiber heating layer 696 can be aligned with the NIR light source 352. The NIR light source 352 can have a third surface area that approximately matches the second surface area of the second carbon fiber heating layer 696.

As shown and described, the combination of the first carbon fiber heating layer 694 and the second carbon fiber heating layer 696 is configured to evenly heat the lumbosacral region (for example, the lumbar region 104 and the sacral region 108) of the back 106 of the user 100. If the second carbon fiber heating layer 696 were not included in the carbon fiber heating portion 478, even heating of the lumbosacral region could not be achieved because the NIR light source 352 absorbs some of the heat generated by the carbon fiber heating portion 478. Thus, if the second carbon fiber heating layer 696 were not included, the region of the strap 210 corresponding to the NIR light source 352 would provide less heat to the back 106 of the user 100 than the surrounding portions. To mitigate this issue, the second carbon fiber heating layer 696 is placed between the first carbon fiber heating layer 694 and the NIR light source 352 and comprises a size and shape (and thus, a surface area) that approximately matches those of the NIR light source 352.

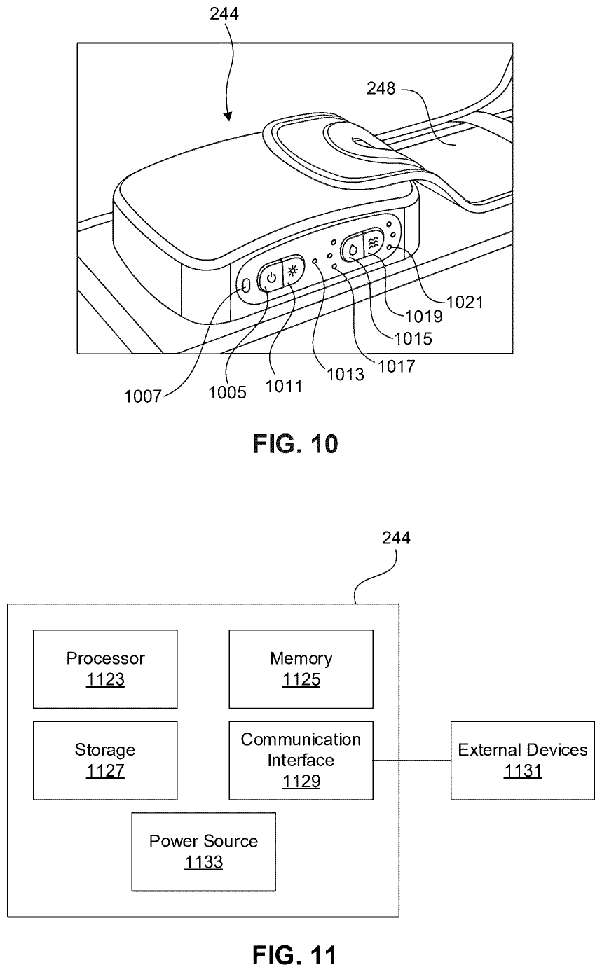

The second carbon fiber heating layer 696 is configured to provide additional heat aligned with the NIR light source 352. For example, the first carbon fiber heating layer 694 can be configured to draw a first amount of power from a power source within the strap (further described with reference to FIG. 10), and the second carbon fiber heating layer 696 can be configured to draw a second amount of power from the power source within the strap. In some embodiments, the first amount of power is different than the second amount of power. In an example embodiment, the first amount of power can be approximately 30 Watts (W) and the second amount of power can be approximately 5 W. The power drawn by the first carbon fiber heating layer 694 and the second carbon fiber heating layer 696 can cause the first carbon fiber heating layer 694 and the second carbon fiber heating layer 696 to increase in temperature. For example, the first carbon fiber heating layer 694 can reach a first temperature of approximately 39 degrees Celsius and the second carbon fiber heating layer 696 can reach a second temperature of approximately 6 degrees Celsius. The NIR light source 352 absorbs heat such that, if the second carbon fiber heating layer 696 were not present, the temperature of the central portion 212 would not be evenly distributed across the back 106. For example, in the region of the NIR light source 352, the temperature would decrease based on the amount of heat absorbed by the NIR light source 352. The additional heat provided by the second carbon fiber heating layer 696 can thus be absorbed by the NIR light source 352 such that the temperature of the central portion 212 in the region of the NIR light source 352 approximately matches the temperature of the central portion 212 in regions outside of the NIR light source 352. Accordingly, the NIR light source 352 absorbs at least some of the heat provided by a combination of the first carbon fiber heating layer 694 and the second carbon fiber heating layer 696 such that the combination of the first carbon fiber heating layer 694 and the second carbon fiber heating layer 696 evenly heats the central portion 212.

FIG. 9 is a perspective view of a lumbar support element 901 according to embodiments of the present disclosure. In some embodiments, the lumbar support element 901 can be removably coupled to the central portion 212. In some embodiments, the lumbar support element 901 can be removably coupled to the central portion 212 by inserting lumbar support element 901 in opening 213 of the central portion 212. In some embodiments, the central portion 212 might not include opening 213, and the lumbar support element 901 can be secured within the central portion 212 such that the lumbar support element 901 cannot be removed without damaging the central portion 212. The lumbar support element 901 can be located further from the back 106 of the user 100 than one or more of the NIR light source 352, the carbon fiber heating portion 478, or the vibration elements 462 when the strap 210 is worn by the user 100. For example, the lumbar support element 901 can be located further from the back 106 of the user 100 than the NIR light source 352, the carbon fiber heating portion 478, and the vibration elements 462. Arranged as described, the lumbar support element 901 can serve to maintain contact between the central portion 212 and the back 106 of the user 100 and avoid areas of non-contact.

In some embodiments, the lumbar support element 901 can comprise a contoured portion 903 configured to follow a contour of the lumbar region 104 and the sacral region 108 of the back 106 of the user 100. In some embodiments, the contoured portion 903 can help maintain contact between the central portion 212 and the back 106 of the user 100.

FIG. 10 is an illustration of the controller 244 according to embodiments of the present disclosure. The controller 244 is configured to control operation of the therapeutic device 102 to provide one or more therapy modalities to the user 100. In some embodiments, the controller 244 may include one of a hook portion or a loop portion, such that the first tightening strap 246 or the second tightening strap 248 may be coupled to a portion of the controller 244 in a hook and loop connection. In some embodiments, attaching the first tightening strap 246 or the second tightening strap 248 to the portion of the controller 244 does not obscure the view of the buttons and light indicators of the controller 244 from the user 100.

The controller 244 comprises various buttons for manipulation by the user 100 and indicators to show the user 100 the status of the therapeutic device 102. For example, the controller 244 can comprise a power button 1005 that the user 100 can press to turn the therapeutic device 102 on or off. When the therapeutic device 102 is on, a battery indicator 1007 can indicate a current power level of the therapeutic device 102. For example, the battery indicator 1007 can illuminate green when the battery charge is greater than or equal to 70 percent of a full charge. The battery indicator 1007 can illuminate orange when the battery charge is greater than or equal to 20 percent and less than 70 percent of a full charge. The battery indicator 1007 can illuminate red when the battery charge is less than or equal to 20 percent of a full charge.

In some embodiments, the battery can be charged during use of the therapeutic device 102. For example, if the user plugs in the therapeutic device 102 while operating the therapeutic device 102, the battery can charge while the therapeutic device 102 is in use. In some embodiments, a user may charge the battery of the therapeutic device 102 while operating the therapeutic device 102 if using a higher power adapter or charger. In some embodiments, the battery cannot be charged during use of the therapeutic device 102 if using a lower power adapter or charger in order to prevent overheating of the therapeutic device 102. In such embodiments, if the user plugs in the therapeutic device 102 while operating the therapeutic device 102, power can be provided to operate the therapeutic device 102 but not to charge the battery.

The controller 244 can comprise a NIR light button 1011. The user 100 can press the NIR light button 1011 to operate the NIR light source 352. In some embodiments, the NIR light button 1011 can be pressed multiple times to provide different durations of NIR light therapy. In embodiments where the NIR light therapy is not initiated upon turning the power on, the user 100 can press the NIR light button 1011 once to initiate NIR light therapy for a duration of approximately 20 minutes. In embodiments in which the NIR light therapy is initiated upon turning the power on, the NIR light therapy is initiated for the approximate 20-minute duration without pressing the NIR light button 1011. The user 100 can press the NIR light button 1011 (again, in instances where the user 100 pressed the NIR light button 1011 to initiate the NIR light therapy) to initiate NIR light therapy for a duration of approximately 10 minutes. The user can press the NIR light button again to turn off the NIR light source 352. Though two NIR light therapy durations are disclosed, more or fewer NIR light therapy durations can be implemented. The controller 244 can comprise a NIR indicator 1013. When the NIR light source 352 is operating, the NIR indicator 1013 can be illuminated (for example, an orange indicator light) to notify the user 100 that the NIR light source 352 is providing NIR light therapy. In some embodiments, the different durations of NIR light therapy can be indicated by, for example, a different color illumination, a different intensity of illumination, or a blinking illumination.

The controller 244 can comprise a heat button 1015. The user 100 can press the heat button 1015 to operate the carbon fiber heating portion 478 (and thus, the FIR light source 482). In some embodiments, the heat button 1015 can be pressed multiple times to provide different levels of heat therapy. In embodiments in which the heat therapy is not initiated upon turning the power on, the user 100 can press the heat button 1015 once to initiate heat therapy at a maximum temperature (for example, approximately 45 degrees Celsius). In embodiments in which the heat therapy is initiated upon turning the power on, the heat therapy is initiated at the maximum temperature without pressing the heat button 1015. The user 100 can press the heat button 1015 (again, if the user 100 pressed the heat button 1015 to initiate the heat therapy) to initiate heat therapy at a medium temperature (for example, approximately 42 degrees Celsius). The user 100 can press the heat button 1015 again to initiate heat therapy at a minimum temperature (for example, approximately 39 degrees Celsius). The user 100 can press the heat button 1015 again to turn off the carbon fiber heating portion 478 (and thus, the FIR light source 482). Though three temperature levels are disclosed, more or fewer temperature levels can be implemented. The controller 244 can comprise a heat indicator 1017. In some embodiments, the heat indicator 1017 can comprise a number of indicator lights that matches the number of temperature levels. For example, the heat indicator 1017 can comprise three indicator lights based on the above example of a maximum, medium, and minimum temperature. When the therapeutic device 102 is providing the maximum temperature, all three indicator lights can be illuminated. When the therapeutic device 102 is providing the medium temperature, two of the three indicator lights can be illuminated. When the therapeutic device 102 is providing the minimum temperature, one of the three indicator lights can be illuminated. When the therapeutic device 102 is not providing heat therapy, none of the indicator lights can be illuminated.

The controller 244 can comprise a vibration button 1019. The user can press the vibration button 1019 to operate the vibration elements 462. In some embodiments, the vibration button 1019 can be pressed multiple times to provide different levels of vibration therapy. In embodiments in which the vibration therapy is not initiated upon turning the power on, the user 100 can press the vibration button 1019 once to initiate vibration therapy at a maximum frequency (for example, approximately 100 Hz). In embodiments in which the vibration therapy is initiated upon turning the power on, the vibration therapy is initiated at the maximum vibration frequency without pressing the vibration button 1019. The user 100 can press the vibration button 1019 (again, if the user pressed the vibration button 1019 to initiate the vibration therapy) to initiate vibration therapy at a medium frequency (for example, approximately 75 Hz). The user 100 can press the vibration button 1019 again to initiate vibration therapy at a minimum frequency (for example, approximately 50 Hz). The user can press the vibration button 1019 again to turn off the vibration elements 462. Though three vibration frequencies are disclosed, more or fewer vibration intensity levels can be implemented. In some embodiments, the user may press the vibration button 1019 a predetermined number of times to initiate different patterns of the vibration therapy, such as a wave pattern, a pulse pattern, or a constant pattern of vibration of the vibration elements 462. The controller 244 can comprise a vibration indicator 1021. In some embodiments, the vibration indicator 1021 can comprise a number of indicator lights that matches the number of vibration frequencies. For example, the vibration button 1019 can comprise three indicator lights based on the above example of a maximum, medium, and minimum vibration frequency. When the therapeutic device 102 is providing the maximum vibration frequency, all three indicator lights can be illuminated. When the therapeutic device 102 is providing the medium vibration frequency, two of the three indicator lights can be illuminated. When the therapeutic device 102 is providing the minimum vibration frequency, one of the three indicator lights can be illuminated. When the therapeutic device 102 is not providing vibration therapy, none of the indicator lights can be illuminated. In some embodiments, the number of indicator lights illuminated can correspond to which of the different vibration therapy patterns has been selected by the user pressing the vibration button 1019 (e.g., illumination of one light indicator can correspond to a constant vibration pattern, illumination of two light indicators can correspond to a pulse vibration pattern, and illumination of three light indicators can correspond to a wave vibration pattern).

In some embodiments, the therapeutic device 102 can be configured to initiate all therapies at the maximum level when the therapeutic device 102 is turned on (by pressing the power button 1005). The user 100 can change the type of therapy and/or intensity of the therapy by pressing the buttons corresponding to the desired therapy until the desired combination is achieved. In some embodiments, the therapeutic device 102 can be configured to turn off automatically after a predetermined duration. For example, the therapeutic device 102 can turn off automatically after operating for approximately 30 minutes. In some embodiments, each therapy can be turned off automatically after a predetermined duration. For example, the NIR light source 352 can be configured to automatically shut off after approximately 10 minutes of operation. As another example, the carbon fiber heating portion 478 can be configured to automatically shut off after approximately 20 minutes of operation. As yet another example, the vibration elements 462 can be configured to automatically shut off after approximately 20 minutes of operation.

FIG. 11 is a block diagram of the controller 244 of FIG. 10 according to embodiments of the present disclosure. The controller 244 can be used to implement the systems and methods disclosed herein. For example, the controller 244 can receive data related to operation of the therapeutic device 102 and operate the therapeutic device 102 accordingly. In an example hardware configuration, the controller 244 generally includes a processor 1123, a memory 1125, a storage 1127, and a communication interface 1129. The processor 1123 can be any suitable processor, such as a central processing unit, for executing computer instructions and performing operations described thereby. The memory 1125 can be a volatile memory, such as random-access memory (RAM). The storage 1127 can be a non-volatile storage device, such as a hard disk drive (HDD) or a solid-state drive (SSD). The storage 1127 can form a computer readable medium that stores instructions (e.g., code) executed by the processor 1123 for operating the therapeutic device 102, for example, in the manners described above and below. The communication interface 1129 is in communication with, for example, external devices 1131, for sending to and receiving from various signals (for example, control signals and/or notifications). The external devices 1131 can include, for example, one or more sensors coupled to the central portion 212 and/or a mobile device (for example, a mobile device associated with the user 100). The controller 244 may control operation of the therapeutic device 102 based on signals and/or instructions received from the external devices 1131.

For example, the one or more sensors coupled to the central portion 212 can include temperature sensors (for example, thermocouples or thermistors). The temperature sensors can be distributed across the central portion 212 such that the controller 244 can receive temperature data from the temperature sensors and can adjust an amount of power provided to the carbon fiber heating portion 478 based on the temperature sensor data. More specifically, the controller 244 can adjust an amount of power provided to the carbon fiber heating portion 478 to increase or decrease the temperature of the carbon fiber heating portion 478 based on the temperature data from the temperature sensors.

As another example, the one or more sensors can include proximity sensors. In some embodiments, the user 100 may wear the therapeutic device 102 over clothing. In such embodiments, the proximity sensors can determine that the central portion 212 is spaced apart from the back 106 (by, for example, the clothing). Based on the distance between the back 106 and the central portion 212, the controller 244 can provide power to the carbon fiber heating portion 478 such that the temperature experienced by the user 100 when the therapeutic device 102 is worn over clothing is similar to the temperature experienced by the user 100 when the therapeutic device 102 is worn against the skin.

As another example, the external devices 1131 can comprise a mobile device (for example, a mobile device belonging to the user 100) configured to communicate with the controller 244. In some embodiments, the communication interface 1129 can be configured to connect with the mobile device via, for example, a Bluetooth connection. In such embodiments, the user 100 can control the therapeutic device 102 using the mobile device. For example, the mobile device may include an application that provides the user 100 the ability to change settings (for example, temperature, vibration frequency, etc.) by interacting with the application, and the changes made by the user 100 can be sent to the controller 244 via the Bluetooth connection. The controller 244 can then execute the desired changes made by the user 100. By connecting the therapeutic device 102 to a mobile device application, the user 100 can use the application to personalize treatment on the therapeutic device 102 by selecting specific treatment durations, temperatures, localization, intensities, and patterns. In some embodiments, the application can prompt the user 100 to use the therapeutic device 102 at certain times of the day relevant to the user's daily activity and track use of the therapeutic device 102 and treatment outcomes to allow optimization of the therapeutic device 102 to treat the specific needs of the user 100.

The controller 244 can comprise a power source 1133. In some embodiments, the power source 1133 can be a battery that is electrically coupled to each of the treatment modalities described herein. In some embodiments, the battery can be a removable battery. In some embodiments, the battery can be secured within the controller 244 and cannot be removed without damaging the controller 244. In either implementation, the battery can be rechargeable. In some embodiments, securing the battery within the controller 244 may improve battery reliability. In some embodiments, the power source 1133 can provide power to operate the device for various durations depending on the operating level. For example, the power source 1133 can be configured to power the therapeutic device 102 for approximately 105 minutes when operating the therapeutic device 102 at high operation levels (for example, high temperature, high vibration frequency, etc.). In some embodiments, the power source 1133 can be configured to power the therapeutic device 102 for approximately 145 minutes when operating the therapeutic device 102 at medium operation levels (for example, medium temperature, medium vibration frequency, etc.). In some embodiments, the power source 1133 can be configured to power the therapeutic device 102 for approximately 170 minutes when operating the therapeutic device 102 at low operation levels (for example, low temperature, low vibration frequency, etc.).

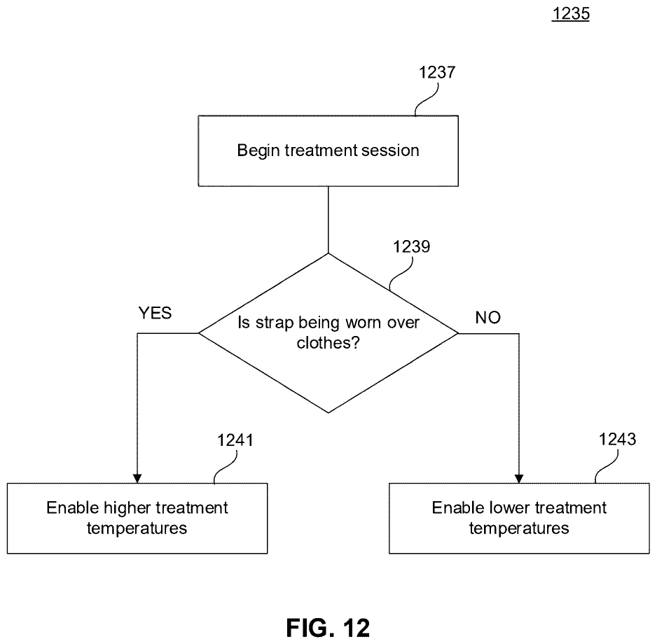

FIG. 12 is a flowchart of a method 1235 to determine a treatment temperature according to embodiments of the present disclosure. The method 1235 can be implemented, at least in part, by the controller 244.

At step 1237, a treatment session is started. For example, the user 100 can start the treatment session by pressing the power button 1005. In some embodiments, pressing the power button 1005 can initiate NIR light therapy via the NIR light source 352, heat therapy and FIR light therapy via the carbon fiber heating portion 478 and FIR light source 482, and vibration therapy via the vibration elements 462. In some embodiments, each of the therapy modalities initiated can be initiated at their maximum levels and can be changed by the user 100. The user can change the therapy modalities by pressing a button corresponding to the desired therapy modality on the controller 244. For example, the user 100 can press the NIR light button 1011 to change the NIR light therapy setting. The user can press the heat button 1015 to change the temperature and FIR light therapy setting. The user 100 can press the vibration button 1019 to change the vibration therapy setting.

Alternatively, the user 100 can adjust the settings using an application on a mobile device associated with the user. For example, the mobile device can connect to the controller 244 from the application via a Bluetooth connection. The user 100 can enter the desired settings on the application, and the desired settings can be sent to the controller 244 via the Bluetooth connection. The controller 244 can implement the desired settings and operate the therapeutic device 102 according to the desired settings.

At step 1239, a determination is made regarding whether the strap 210 is being worn over clothing. For example, one or more proximity sensors located on the central portion 212 can determine whether the user 100 is wearing the strap 210 over clothing based on a distance between the one or more proximity sensors and the skin of the user 100. Alternatively, the application on the mobile device can display a prompt to the user 100 and ask the user 100 if the strap 210 is being worn over clothing.

If the user 100 replies affirmatively that the strap 210 is being worn over clothing (or if the controller 244 determines that the strap 210 is being worn over clothing based on the one or more proximity sensors), then at step 1241, higher treatment temperatures are enabled. For example, a maximum treatment temperature that the therapeutic device 102 can be permitted to reach if worn on the skin can be 45 degrees Celsius, and a maximum treatment temperature that the therapeutic device 102 can be permitted to reach if worn over clothing can be 48 degrees Celsius. Thus, when the determination is made that the therapeutic device 102 is being worn over clothing, the maximum treatment temperature of 45 degrees Celsius can be enabled.

If the user 100 replies negatively that the strap 210 is being worn over clothing (or if the controller 244 determines that the strap 210 is being worn against the skin based on the one or more proximity sensors), then at step 1243, lower treatment temperatures (for example, the maximum treatment temperature of 45 degrees Celsius) are enabled.

In some embodiments, the application on the mobile device can provide the user 100 with various options that may not be available when operating the device via the controller 244. For example, the application can provide the user the option to increase a duration of temperature therapy. More specifically, a default duration for temperature therapy when selected via the controller 244 can be 20 minutes. When selecting the temperature therapy on the application, the user 100 can have the option of selecting a duration of up to 90 minutes. In such embodiments the application can recommend a lower temperature (if applicable) for the longer duration.

Arranged and operated as described, the therapeutic device 102 can provide numerous benefits for the user 100. For example, the combination of therapy modalities provided by the therapeutic device 102 (NIR light therapy, heat therapy, FIR light therapy, and vibration therapy) can reduce pain (for example, lower back pain) and promote healing (for example, by promoting cellular repair and regeneration of tissue). The therapeutic device 102 can also aid in reducing rehabilitation and recovery time (for example, from injury, surgical procedures, etc.). Additionally, the therapeutic device 102 can improve mobility and range of motion and reduce soreness, stiffness, and tension. More specific to the disclosure above, the therapeutic device 102 can be used to treat chronic lower back pain, non-specific lower back pain, and strains of back muscles. At least some of the lower back pain that can be treated by the therapeutic device 102 can be related to conditions such as sciatica, scoliosis, and herniated disks.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Aspects using the singular or plural number may also include the plural or singular number respectively. The word “or” in reference to a list of two or more items, covers all of the following interpretations of the word: any of the items in the list, all of the items in the list, and any combination of the items in the list.

The above-detailed description of aspects of the disclosure is not intended to be exhaustive or to limit the teachings to the precise form disclosed above. While specific aspects of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another aspect, instructions or sub-operations of distinct operations may be implemented in an intermittent and/or alternating manner.

The teachings of the disclosure provided herein can be applied to other systems, not necessarily the system described above. The elements and acts of the various aspects described above can be combined to provide further aspects. Any measurements or dimensions described or used herein are merely exemplary and not a limitation on the present disclosure. Other measurements or dimensions are within the scope of the disclosure.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further aspects of the disclosure.

These and other changes can be made to the disclosure in light of the above Detailed Description of the Aspects. While the above description describes certain aspects of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific aspects disclosed in the specification unless the above Detailed Description of the Aspects section explicitly defines such terms. Accordingly, the actual scope of the disclosure encompasses not only the disclosed aspects, but also all equivalent ways of practicing or implementing the disclosure under the claims.

Accordingly, although exemplary aspects of the disclosure have been shown and described, it is to be understood that all the terms used herein are descriptive rather than limiting, and that many changes, modifications, and substitutions may be made by one having ordinary skill in the art without departing from the spirit and scope of the disclosure.

1. A wearable therapeutic device, comprising:
a strap configured to be worn around a lumbar region of a back of a user, the strap comprising:
a central portion comprising a central longitudinal axis extending through a center of the central portion and oriented approximately perpendicular to a spine of the user when the strap is worn by the user;
a first end portion extending from a first side of the central portion, the first end portion comprising a first longitudinal axis extending through a center of the first end portion and oriented approximately perpendicular to the spine of the user when the strap is worn by the user; and
a second end portion extending from a second side of the central portion opposite the first side of the central portion, the second end portion comprising a second longitudinal axis extending through a center of the second end portion and oriented approximately perpendicular to the spine of the user when the strap is worn by the user;
wherein the first longitudinal axis and the second longitudinal axis are approximately coaxial with each other, and the central longitudinal axis is located further from a head of the user than the first longitudinal axis and the second longitudinal axis when the strap is worn by the user;
a far-infrared light source located in the central portion;
a near-infrared light source located in the central portion and configured to contact the back of the user when the strap is worn by the user, wherein the near-infrared light source is positioned between the back of the user and the far-infrared light source when the strap is worn by the user;
at least one vibration element located in the central portion; and
a heating element located in the central portion.

2. The wearable therapeutic device of claim 1, wherein the heating element comprises a carbon fiber heating portion, the carbon fiber heating portion comprising carbon fiber strands arranged in a pattern.

3. The wearable therapeutic device of claim 2, wherein the pattern comprises a sinusoidal pattern.

4. The wearable therapeutic device of claim 1, wherein the heating element comprises a first carbon fiber layer and a second carbon fiber layer, wherein the first carbon fiber layer is larger than the second carbon fiber layer, and the second carbon fiber layer is located closer to the back of the user when the strap is worn by the user.

5. The wearable therapeutic device of claim 4, wherein the second carbon fiber layer is located between the near-infrared light source and the first carbon fiber layer, and the second carbon fiber layer has approximately a same size and shape as the near-infrared light source.

6. The wearable therapeutic device of claim 5, wherein a combination of the first carbon fiber layer and the second carbon fiber layer is configured to evenly heat a lumbosacral region of the back of the user.

7. The wearable therapeutic device of claim 6, wherein the first carbon fiber layer draws a first amount of power from a power source within the strap and the second carbon fiber layer draws a second amount of power from the power source, and the first amount of power is different than the second amount of power.

8. The wearable therapeutic device of claim 7, wherein the first carbon fiber layer and the second carbon fiber layer are configured to generate heat at the central portion, wherein the first carbon fiber layer reaches a first temperature when the first carbon fiber layer draws power from the power source within the strap to generate a first portion of the heat, and the second carbon fiber layer reaches a second temperature when the second carbon fiber layer draws power from the power source to generate a second portion of the heat,
wherein the near-infrared light source is configured to absorb at least some of the heat such that the combination of the first carbon fiber layer and the second carbon fiber layer evenly heats the central portion.

9. A wearable therapeutic device, comprising:
a strap configured to be worn around a lumbar region of a back of a user, the strap comprising:
a central portion;
a first end portion extending from a first side of the central portion; and
a second end portion extending from a second side of the central portion opposite the first side of the central portion;
at least one vibration element located in the central portion;
a near-infrared light source located in the central portion and configured to contact the back of the user when the strap is worn by the user;
a carbon fiber heating portion located in the central portion, wherein the carbon fiber heating portion is located between the at least one vibration element and the near-infrared light source when the strap is worn by the user such that the near-infrared light source is located closer to the back of the user than the at least one vibration element when the strap is worn by the user, wherein the carbon fiber heating portion emits far-infrared light when the carbon fiber heating portion receives power from a power source in the strap; and
a housing configured to secure the at least one vibration element, the near-infrared light source, and the carbon fiber heating portion.

10. The wearable therapeutic device of claim 9, wherein the carbon fiber heating portion is configured to evenly heat a lumbosacral region of the back of the user.

11. The wearable therapeutic device of claim 10, wherein the carbon fiber heating portion comprises a first carbon heating fiber layer adjacent to a second carbon fiber heating layer, wherein the first carbon fiber heating layer and the second carbon fiber heating layer are different sizes.

12. The wearable therapeutic device of claim 9, wherein the carbon fiber heating portion is configured to emit a first dose of far-infrared light when the carbon fiber heating portion is maintained at a first temperature for a first duration, and the carbon fiber heating portion is configured to emit a second dose of far-infrared light when the carbon fiber heating portion is maintained at a second temperature for a second duration, wherein the first dose is different than the second dose and the first temperature is different than the second temperature.

13. The wearable therapeutic device of claim 11, wherein the first carbon fiber heating layer comprises a first surface area and the second carbon fiber heating layer comprises a second surface area, wherein the first surface area is at least twice as large as the second surface area.

14. The wearable therapeutic device of claim 13, wherein the second carbon fiber heating layer is aligned with the near-infrared light source, wherein the near-infrared light source comprises a third surface area that approximately matches the second surface area of the second carbon fiber heating layer.

15. A wearable therapeutic device, comprising:
a strap configured to be worn around a lumbar region of a back of user, the strap comprising:
a central portion comprising a top edge and a bottom edge, wherein the top edge of the central portion is located closer to a head of the user when the strap is worn by the user;
a first end portion extending from a first side of the central portion, the first end portion comprising a top edge and a bottom edge, wherein the top edge of the first end portion is located closer to the head of the user than the bottom edge of the first end portion when the strap is worn by the user; and
a second end portion extending from a second side of the central portion opposite the first side, the second end portion comprising:
a top edge and a bottom edge, wherein the top edge of the second end portion is located closer to the head of the user than the bottom edge of the second end portion when the strap is worn by the user; and
a controller coupled to the second end portion configured to be located in front of the user when the strap is worn by the user,
wherein a first non-zero distance between the top edge of the central portion and the top edges of the first end portion and the second end portion in a direction approximately parallel to a spine of the user is smaller than a second non-zero distance between the bottom edge of the central portion and the bottom edges of the first end portion and the second end portion in the direction approximately parallel to the spine of the user;
at least one vibration element located in the central portion and electrically coupled to the controller;
a heating element located in the central portion and electrically coupled to the controller;
a near-infrared light source located in the central portion and electrically coupled to the controller; and
a far-infrared light source located in the central portion and electrically coupled to the controller,
wherein the controller is configured to operate the at least one vibration element, the heating element, the near-infrared light source, and the far-infrared light source.

16. The wearable therapeutic device of claim 15, wherein the heating element comprises a first carbon fiber heating layer and a second carbon fiber heating layer, wherein the second carbon fiber heating layer is adjacent to the first carbon fiber heating layer and comprises a smaller surface area than the first carbon fiber heating layer.

17. The wearable therapeutic device of claim 16, wherein the controller is configured to cause the first carbon fiber heating layer and the second carbon fiber heating layer to generate heat at the central portion, wherein the controller is further configured to cause first carbon fiber layer to reach a first temperature to generate a first portion of the heat, and wherein the controller is configured to cause the second carbon fiber heating layer to reach a second temperature to generate a second portion of the heat, the first temperature being different than the second temperature,
wherein the near-infrared light source is configured to absorb at least some of the heat such that a combination of the first carbon fiber heating layer and the second carbon fiber heating layer evenly heats the central portion.

18. The wearable therapeutic device of claim 16, wherein the far-infrared light source comprises the heating element, and the controller is configured to cause the far-infrared light source to emit a first dose of far-infrared light when the heating element is maintained at a first temperature for a first duration, and the controller is configured to cause the far-infrared light source to emit a second dose of far-infrared light when the heating element is maintained at a second temperature for a second duration, wherein the first dose is different than the second dose and the first temperature is different than the second temperature.

19. The wearable therapeutic device of claim 18, wherein the first dose of far-infrared light is greater than or equal to 8 Joules per square centimeter and less than or equal to 10 Joules per square centimeter when the first temperature is approximately 39 degrees Celsius.

20. The wearable therapeutic device of claim 18, wherein the second dose of far-infrared light is greater than or equal to 10 Joules per square centimeter and less than or equal to 12 Joules per square centimeter when the second temperature is approximately 42 degrees Celsius.
